(19) United States
(12) Patent Application Publication
(10) Pub. No.: US 2011/0300624 A1
(43) Pub. Date: Dec. 8, 2011

(54) PHOTOBIOREACTOR

(76) Inventor: Kian Wee Lim @ Tian Kian Wee, Johor Darul Takzim (MY)

(21) Appl. No.: 13/128,652

(22) PCT Filed: Oct. 30, 2009
(86) PCT No.: PCT/MY2009/000177

§ 371 Date: Aug. 24, 2011

(30) Foreign Application Priority Data
Nov. 12, 2008 (MY) PI 20084541

(51) Int. Cl. C12M 1/14

(52) U.S. Cl. 435/292.1

(57) ABSTRACT

A photobioreactor (100) comprises a base (110); a supportive frame (120) extending upwardly from the base; a plurality of trays (130) for culturing phototropic microorganisms, ranking vertically from a uppermost tray to a bottommost tray, supported co-axially on the supportive frame (120) and spaced apart one and other in a predetermined gap at the vertical plane to optimize exposure to a light source; and a protective member (140) located on top of the uppermost tray by mounting onto the supportive frame (120) wherein the plurality of trays (130) and the protective member (140) are made of light permeable material.

### FIELD OF INVENTION

The present invention relates to a photobioreactor to be used for cultivation of phot[sigma]tropic microorganisms to produce useful compounds'. In more, specific, the disclosed photobioreactor employs a trays-stacking method to cultivate phototropic microorganisms in which, the photobioreactor is integrated with closed, semi-closed, and open type cultivation system thus promoting phototropic microorganisms growth by utilizing carbon dioxide and light energy effectively and efficiently with limited land area available.

### BACKGROUND OF THE INVENTION

Light is essential for most of the phototropic microorganisms such as photo synthetic bacteria or microalgae to grow in almost every type of photobioreactors. Current limiting factors impeding phototoopic microorganisms growth in photobioreactors are the availability of light and the input of carbon dioxide. Most of the photobioreactors have limited yield for the cultivation due to limited availability of light at the inner part of the photobioreactor as light is unable to penetrate through to the inner section of the photobioreactor. Apart from that, most photobioreactor employ a closed system for phototropic microorganism cultivation to prevent contamination from some airborne particles and undesired growth such as other microorganisms. Nevertheless, such approach is successful not without paying a price that reduced carbon dioxide and increased oxygen concentration in the culture medium. Due to the closed environment, increased oxygen concentration often causes photo-respiration that reduce growth rate, while limited amount of carbon dioxide in the culture medium restricts the photo synthetic activities of phototropic microorganisms. Consequently, alternative carbon dioxide source and oxygen remover may be made to associate with the cultivation system, recurring extra operating cost.

In U.S. patent application Ser. No. 5,162,051, a photobioreactor is disclosed which containing a plurality of baffles mounted on in the photobioreactor tank forming hollow cavities for insertion of light source. Forth provide another photobioreactor capable of semi-continuously exposing the cultivated biomass to a light source via a circulating means in U.S. patent application Ser. No. 5,846,816.

Further U.S. patent application Ser. No. 6,509,188 provides a chamber of photobioreactor with increased sidewall surface greater than planar enveloping surface as found in conventional photobioreactor.

Another international patent application no WO 2005068605 claims a photobioreactor using one or more movable collimators to introduce sufficient light into the culture medium containing the cultivated microorganisms.

### SUMMARY OF THE INVENTION

The present invention aims to provide an apparatus, a photobioreactor, for cultivation of microalgae in a closed, semi-closed, or open system to produce microalgae culture in high purity.

Further object of the present invention is to provide a photobioreactor capable of producing large amount of microalgae per available land space with limited energy used thus lowering the microalgae production cost compared to closed type photobioreactor, which high concentration of CO2 or oxygen remover is needed.

When coupled with open pond such as raceway pond, this photobioreactor permits increase microalgae purity and productivity without additional land usage.

Still another object of the present invention is to optimize the harvest of light, preferably sunlight, for cultivation of microalgae in the disclosed photobioreactor. The disclosed photobioreactor allow light or solar radiations to reach the surface of the photobioreactor through various angles of incident, permitting efficient light energy harvesting by the microalgae. At least one of the preceding objects is met, in whole or in part, by the present invention, in which one of the embodiment of the present invention involves a photobioreactor comprising a base; a supportive frame extending upwardly from the base; a plurality of trays for microalgae cultivation supported co-axially on the supportive frame and spaced apart one and other in a predetermined gap at the vertical plane to optimize exposure to light; and a protective member located on top of the plurality of microalgae cultivation tray by mounting onto the supportive frame wherein the plurality of microalgae cultivation tray and the protective member are made of light permeable material.

Another aspect of the invention may further comprise one or more conduit associated with the trays to drain off the yielded microalgae or supply the necessary nutrient or culture medium into the tray.

In further aspect of the present invention, each tray further comprises a light permeable covering member to<'>cover the tray aiming to reduce the chances for the microalgae culture being exposed to any available contaminants.

In order to achieve higher yielding, the operating parameters of the photobioreactor have to be maintained within a narrow range. Therefore, it is necessary for the present invention includes a means for detecting or monitoring physiochemical parameter of microalgae cultivation in the trays.

Moreover, the trays maybe detachable from the supportive frame for cleaning purpose.

In the preferred embodiment, the disclosed photobioreactor may be coupled with an open pond cultivation system. Hence, portability of the disclosed photobioreactor to the open pond system can be acquired through a means for transportation mounted below the base rendering mobility to the photobioreactor. To harvest additional amount of light for the cultured phototropic microorganism, a specular reflective member is positioned below the plurality of tray for light reflection to reflect light source to the bottommost tray. Possibly, an artificial light source is attached above the uppermost tray or below the bottommost tray to emitting light for growing the microalgae.

Further embodiment of the present invention may have a means for circulating to circulate the culturing phototropic microorganism in the tray.

### BRIEF DESCRIPTION OF THE DRAWINGS

FIG. 1 shows a perspective 3D view of one embodiment of the present invention; and

FIG. 2 shows the cross-sectional view of the embodiment as shown in FIG. 1.

### DETAILED DESCRIPTION OF THE INVENTION

It is to be understood that the present invention may be embodied in other specific forms and is not limited to the sole embodiment described herein. However modification and equivalents of the disclosed concepts such as those which readily occur to one skilled in the art are intended to be included within the scope of the claims which are appended thereto.

The term “phototropic microorganisms” used throughout the description herein refers to microorganisms which capable of harvesting light energy or solar energy for initialising chemical reactions within the microorganisms to produce chemical compounds. The phototropic microorganisms used herein preferably belong to the cyanobacteria or microalgae species such as Botryococcus sp., Phaeodactyluin sp., Spirulina sp., Hematococcus sp., Nanochloropsis sp., Tetraselmis sp., Chlorella sp., Pleurochysis sp., Dunaliella sp., Scenedesmus sp., etc.

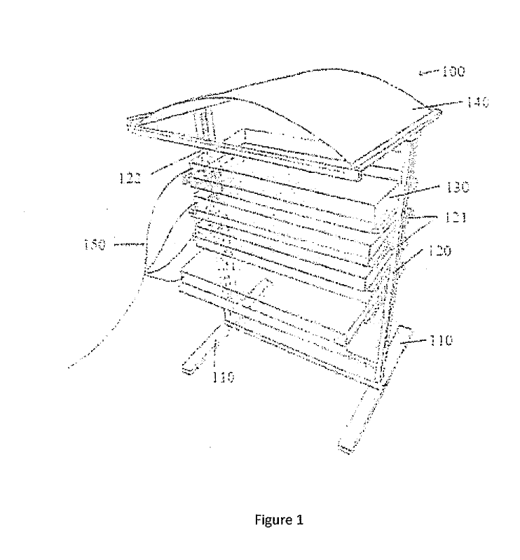

The present invention is a photobioreactor (100), as shown in FIG. 1, comprising a base (110); a supportive frame (120) extending upwardly from the base; and a plurality of trays (130) for cultivation of phototropic microorganisms, ranking vertically from a uppermost tray to a bottommost tray, supported co-axially on the .supportive frame (120)—and spaced apart one and other, in a predetermined gap at the vertical plane to optimize exposure to a light source; wherein the plurality of trays (130) are partially or wholly made of light permeable material. The supportive frame (120) employed in the present invention can adapt different shape and size for accommodating the trays (130). In the preferred embodiment, the supportive frame (120) can be a pair of pole structures (122) which facing oppositely to one another on the base (110). On the surface of the pole structures (122) which facing each other, there is a plurality of projection (121) mounted onto the pole structure (120) to be used for holding and supporting the trays (130). One of such projection (121) on the pole structure (120) shall be coupled with a corresponding projection at similar height level on another pole structure. These projections (121) can be elongated plates crossing the pole structures diagonally at the same planar to the opposition surface of the pole structure (120). Moreover, the pole structure (122) may made to be available for positioning the projection (121) (thus the supported tray as well) at different level in user's preference. For example, a plurality of apertures at different height level can be found on the pole structures (122) for fixing the projection onto the apertures using a screw or nut. Through the supportive frame (120), the trays of the present invention can be stacked vertically one another with a predetermined gap between one another.

Further embodiment of the present invention may have a protective member (140), preferably a transparent sheet, located on top of the uppermost tray by mounting onto the supportive frame (120). Since the tray in the present invention may not be covered during the cultivation and it is necessary to expose the cultivating phototropic microorganisms under sunlight or artificial light for harvesting the energy, contaminants such as water drop or bird feces may fall into the uppermost tray. With the protective member (140), the trays are safeguard from these undesired contaminants. Nonetheless, each of the tray (130) may comprise an individual light permeable covering member to cover the top of the tray in another embodiment to rid off any chances which the cultivating phototropic microorganism in contact with contaminants.

As setting forth, the trays (130) and the protective member (150) may partially or wholly made of light permeable or light transparent material to allow the light energy passing through from the uppermost tray to the bottommost tray. Either in whole or in part which the trays (130) are made of transparent material, it is necessary the bottom of the trays is made of transparent material thus permissible to the light. The light permeable material can be of polymer or glass made, though preferably it is made of plastic or polymer which is light in weight yet mechanically strong enough to support the weight of the culture medium. Still another embodiment of the disclosed photo-bioreactor (100), the trays (130) are detachable from the supportive frame allowing the user to clean the tray or for maintenance purpose.

In order to collect the cultivated product or refill or drain off the tray (130) with culture medium, the present invention employs one or more conduits (150) associated with the trays (130) to conduct the task. These conduits (150) can be used to transfer the cultured phototropic microorganisms at the uppermost tray to the other trays at the lower level. These conduits may also connect with a pumping system for either refilling the tray or draining off the cultivated products. In another aspect, when introducing of additional carbon source into the culture medium is necessary, carbon dioxide can slowly flow through the culture medium via the conduit, or an aerator, to enhance dissolution of the carbon dioxide into the culture medium. When carbon dioxide enriched air or air from the surrounding environment is introduced into the culture medium as the source of carbon dioxide, air filter may be used in the present invention to clean the air particle first to avoid introduction of contaminants into the culture. In the most preferred embodiment, a circulating means is used to continuously mix or circulate the culture medium thus exposing the phototropic microorganism equally to the nutrients and light energy.

Moreover, a stringent cultivating condition is applied to the present invention not only to favor the growth of the desired phototropic species but also prohibit growth of the undesired. Therefore, the physiochemical parameter within the medium is closely monitored through one or more means for detecting or monitoring. Detecting and monitoring devices, preferably sensors, may be used to detect measurements on the physiochemical parameter such as concentration of carbon dioxide, pH, temperature, concentration of nutrients and the like. In accordance with the most preferred embodiment, an optical sensor maybe used to detect density of the cultivated phototropic microorganism in the culture medium. This optical sensor may be coupled with the conduit that the optical sensor may activate the conduit to drain off the culture medium upon detecting the density at certain level and followed by refilling the trays. Preferably, the cultivated phototropic microorganisms can be harvested from the system when the density is above 0.20 gram per liter of culture medium, though most preferred 1.0 g per liter of culture medium. Nonetheless, the density to determine the harvesting criteria may be varied from one species of the phototropic microorganism from another.

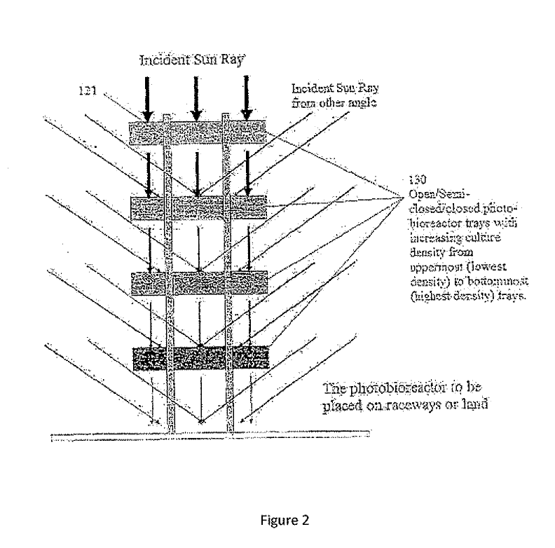

Referring to FIG. 2, it illustrates the mechanisms of light or solar energy being harvested through the present invention. The trays containing the culture medium are stacked on or ranked from bottom to the top of the supportive frame spacing apart at a predetermined gap between the lower tray and the upper tray. Owing to the transparency of the bottom of each tray, the solar energy of the light are able to pass through from the uppermost tray to the bottommost tray while it is preferred that each tray to have different culture densities for effectively harvesting the light energy. In accordance with the most preferred embodiment, the culture densities of the upper trays are always lower than the trays below them to allow higher amount of light penetration through each tray while maximizing light energy utilization efficiency. This forms a culture density gradient across the trays where culture density is the lowest at the uppermost tray and highest at the bottommost tray. Furthermore, the incidental light energy may also radiate into the tray through an inclination angle and the amount of such incidental light entering the tray may be decided by the gap spacing the trays apart. Apart from that, the surface area available to volume of the culture medium needs to maintain in a ratio of 1 cm² to 1.5-10 ml to achieve optimal harvest of light and dissolution of carbon dioxide into the culture medium yet the culture medium is not too deep to prohibit light penetration.

To optimize the energy harvested, a specular reflective member may be positioned below the bottommost tray for light reflection. Thus, any incidental light falls onto the specular reflective member will be reflected upwardly from the bottommost tray towards the uppermost tray.

According to the most preferred embodiment an artificial light source for emitting the light energy can be positioned below the bottommost tray or above the uppermost tray to supply external energy source to the culturing when the solar energy is not available due to environmental factor.

Moreover, in the preferred embodiment, the predetermined gap in between each tray should be at least equal to half the width of the tray so that the incident light or ray from the side come into contact with the culture medium within the tray at least from 45 degree angle at both left and right sides thus harvesting optimal light energy from the surrounding environment. The bigger the predetermined gap between the trays, the more incident light falls into the lower tray. Nevertheless, variable gap distance can be used as well.

In one of the embodiments, the disclosed photo-bioreactor may be rendered with limited mobility by having a means for transportation, wheel-liked members, mounted below the base. It is important to be noted that the cultivation derived from the present invention is in high purity which may then be transferred to an open pond facility for mass culturing which large volume of high concentration of phototropic culture is needed as a starting culture. Equipped with such mobility may provide portability of the present invention from one open pond to another.

In respect to the preferred embodiment disclosed, the user of the present invention may prepare the culture medium containing dissolved nutrients such as nitrates, phosphates, potassium, calcium, magnesium and other trace minerals by adjusting the pH to an appropriate level. The preparation is followed by adding 1 to 30% of microalgae stock culture to the culture medium which is located in the uppermost tray. When the culture density of the microalgae doubles due to growth, for example from 10% of maximum density reaches 20% of maximum density, the microalgae and the cultivation medium in the uppermost tray shall be transferred into the second tray. Respectively, the microalgae culture should be transferred to the bottommost tray or the open cultivation system when the density is 50% above and harvested from the bottommost tray or the open cultivation system once the density reached 100% or optimal density for harvesting. In example given, the starting microalgae culture may begin with 6.25% in the uppermost tray and allowed to grow until the density reaches 12.5%, then it is transferred to second lower tray to grow until the density reaches 25.0%. When the microalgae culture from the second lower tray reaches 50% of maximum culture density in the third lower tray, the microalgae and culture medium may be transferred to the bottommost tray or the open pond such as raceway to culture until the culture density attain 100% or optimal density for harvesting. In another example of preferred cultivation method, the culture density of the microalgae may remain the same for all of the trays.

The present disclosure includes as contained in the appended claims, as well as that of the foregoing description. Although this invention has been described in its preferred form with a degree of particularity, it is understood that the present disclosure of the preferred form has been made only by way of example and that numerous changes in the details of construction and the combination and arrangements of parts may be resorted to without departing from the scope of the invention.

1-10. (canceled)

11. A photobioreactor comprising:
a base;
a supportive frame extending upwardly from the base;
a plurality of trays for culturing phototropic microorganisms in a medium, ranking vertically from an uppermost tray to a bottommost tray, supported co-axially on the supportive frame and spaced apart one and other in a predetermined gap at the vertical plane to optimize exposure to a light source; and
wherein the plurality of trays and the protective member are made of light permeable material.

12. A photobioreactor according to claim 11 further comprising one or more conduit associated with the trays.

13. A photobioreactor according to claim 12, wherein each tray further comprising a light permeable covering member to cover the tray.

14. A photobioreactor according to claim 11 further comprising a means for detecting or monitoring physiochemical parameter of the culture medium or condition available on the trays.

15. A photobioreactor according to claim 11, wherein the trays are detachable from the supportive frame.

16. A photobioreactor according to claim 11, further comprising a means for transportation mounted below the base rendering mobility to the photobioreactor.

17. A photobioreactor according to claim 11, further comprising a specular reflective member positioned below the bottommost tray for light reflection.

18. A photobioreactor according to claim 11 further comprising a means for circulating to circulate the culturing algae in the tray.

19. A photobioreactor according to claim 11 further comprising an artificial light source emitting light below the bottommost tray or above the uppermost tray.

20. A photobioreactor according to claim 11 further comprising a protective member located on top of the uppermost tray by mounting onto the supportive frame.
